(19) United States
(12) Patent Application Publication
(10) Pub. No.: US 2024/0148437 A1
(43) Pub. Date: May 9, 2024

(54) ELECTRONIC DEVICE FOR THE OPERATION OF AN ENDOUROLOGICAL LASER SURGERY SYSTEM AND ASSOCIATED METHOD

(71) Applicant: Coloplast A/S, Humlebaek (DK)

(72) Inventor: Olivier Traxer, Paris (FR)

(21) Appl. No.: 18/278,852

(22) PCT Filed: Feb. 28, 2022
(86) PCT No.: PCT/DK2022/050032

§ 371 Date: Aug. 25, 2023

(30) Foreign Application Priority Data
Feb. 28, 2021 (EP) 21305242.6

(51) Int. Cl. A61B 18/24; A61B 17/00; A61B 18/00

(52) U.S. Cl. CPC A61B 18/24; A61B 17/00; A61B 2017/0019; A61B 2017/00194; A61B 2017/00199; A61B 2018/00505

(57) ABSTRACT

The object of the invention is an electronic device for the operation of an endourological laser surgery system. The endourological laser surgery system comprises an endourological laser surgery device. The electronic device comprises a processor; memory; an interface comprising a display and an input device. The electronic device is configured for the acquisition of a configuration scheme of the endourological laser surgery device. The electronic device is configured for the determination, based on the configuration scheme, of an energy parameter associated with the light emission by the endourological laser surgery device. The energy parameter is determined based on an energy level and a pulse duration for the light emission by the endourological laser surgery device. The electronic device is configured for the representation, on the display, of a first user interface object which is representative of the energy parameter.

The present disclosure relates to the field of endourological laser surgery systems, particularly to an electronic device for the operation of an endourological laser surgery system, and to a method for the operation of an endourological laser surgery system.

### BRIEF DESCRIPTION OF THE DRAWINGS

The characteristics and advantages of the present disclosure will be apparent to a person skilled in the art from the following detailed description, and from the examples disclosed with reference to the attached drawings:

FIG. 1 is a diagram illustrating an exemplary endourological laser surgery system according to the present disclosure,

FIG. 2 is a diagram illustrating an exemplary electronic device for the operation of an endourological laser surgery system according to the present disclosure,

FIG. 3 is a diagram illustrating an exemplary user interface screen for the display of exemplary user interface objects which are representative of operational environments according to the present disclosure,

FIG. 4 is a diagram illustrating an exemplary user interface screen for the display of exemplary user interface objects which are representative of modes associated with a selected environment according to the present disclosure,

FIG. 5 is a diagram illustrating an exemplary user interface screen for the display of exemplary user interface objects which are representative of operational environments according to the present disclosure,

FIG. 6 is a diagram illustrating an exemplary user interface screen for the display of exemplary user interface objects which are representative of modes associated with a selected environment according to the present disclosure,

FIG. 7 is a diagram illustrating an exemplary user interface screen for the display of exemplary user interface objects which are representative of operational environments according to the present disclosure,

FIG. 8 is a diagram illustrating an exemplary user interface screen for the display, inter alia, of the first user interface object, which is representative of the energy parameter, according to the present disclosure,

FIG. 9 is a diagram illustrating an exemplary user interface screen for the display of exemplary user interface objects which are representative of operational environments according to the present disclosure,

FIG. 10 is a diagram illustrating an exemplary user interface screen for the “free” operational environment, according to the present disclosure, and

FIG. 11 is a logic diagram illustrating an exemplary method for the operation of an endourological laser surgery system according to the present disclosure.

### DETAILED DESCRIPTION

Various examples and details are described hereinafter, with reference to the figures, where applicable. It should be observed that the figures may or may not be drawn to scale, and that similar structural elements or functions are represented by identical reference numbers in all the figures. It should also be observed that the figures are intended solely to facilitate the description of examples. The figures are not intended to constitute an exhaustive description of the disclosure, nor any limitation of the scope of disclosure. Moreover, an illustrated example does not necessarily incorporate all the aspects or advantages indicated. An aspect or an advantage described in conjunction with a particular example is not necessarily limited to said example, and may be implemented in any other example, even if it is not illustrated as such, or is not described in an explicit manner.

The figures are schematic and simplified in the interests of clarity, and simply illustrate details which are intended to assist the understanding of the disclosure, whereas other details have been omitted. Overall, the same reference numbers are employed for identical or mutually corresponding components.

The employment of an endourological laser surgery system is executed via a user interface. The laser system according to the present disclosure is a pulsed laser system. The user interface poses difficulties to urologists, in terms of the optimum operation of the laser system. The user interface, in many cases, is highly complex, and results in a sub-optimum use of the laser system. Urologists encounter difficulties in the effective use or parameterization of the laser system. Parameters for the laser system comprise an energy level, a pulse duration of light emission, and an emission frequency. This provides a large number of potential combinations. Confusion exists between combinations of parameters for the laser system and their effects upon the endourological areas to be targeted. It is also difficult for urologists to identify the correct combination required to achieve the desired effect. As a result, endourological laser surgery systems are not presently used under optimum conditions.

It is therefore necessary to develop an electronic device with a user interface which can effectively assist a urologist in the use of the endourological laser surgery system. It is therefore necessary to develop an electronic device with a user interface which can effectively assist a urologist in the use of the endourological laser surgery system, at initialization of the laser system. It is therefore necessary to develop an electronic device with a user interface which can effectively assist a urologist in the use of the endourological laser surgery system, from the initial programming of the laser system through to the achievement of the outcome of laser surgery. It is therefore necessary to develop an electronic device with a user interface which can effectively assist a urologist in the monitoring a use of the endourological laser surgery system. The endourological laser surgery system may be employed for renal, ureteral, prostate, vesical and/or urethral laser surgery.

Accordingly, an object of the invention is an electronic device for the operation and/or assisting a use of an endourological laser surgery system. The endourological laser surgery system comprises an endourological laser surgery device. The electronic device comprises a processor; memory; an interface comprising a display and an input device. The electronic device is configured to obtain a configuration scheme of the endourological laser surgery device. The electronic device is configured to determine, based on the configuration scheme, an energy parameter associated with a light emission by the endourological laser surgery device. The energy parameter is determined based on an energy level and a pulse duration of the light emission by the endourological laser surgery device. The electronic device is configured to display, on the display, of a first user interface object representative of the energy parameter.

The energy parameter may be defined as a parameter, expressed, for example, in Joules, and is associated with a pulse duration of the light emission by the endourological laser surgery device. The energy level may be defined as the intensity of light, which can range from dazzling to very low brightness. The pulse duration may be defined as the duration of light emission associated with a pulse. The energy parameter relates to the internal status of the laser device, and permits the user to execute the correct operation of this technical device. The energy parameter can vary in a dynamic manner, and is detected automatically.

The inventor has discovered that the energy parameter disclosed here is the requisite parameter for the achievement of the desired effect, such as, for example, the incision, fragmentation, or pulverization of a kidney stone. The energy parameter may be considered as the force of energy acting on the area to be targeted. The frequency parameter may be considered as a rate of execution. For example, to use the analogy of a motor car, the energy parameter corresponds to revolutions per minute, and the frequency parameter corresponds to speed. For example, the higher the frequency parameter employed, the more difficult it is to achieve accuracy. The inventor has discovered that the energy parameter, combining the energy level and the pulse duration, is useful, whereas the frequency parameter does no more than regulate the rate of task.

In one or more examples, the energy parameter is generated based on a combination of energy level and impulse duration. In one or more example, the energy parameter is not a power parameter. An advantage of the present disclosure is the support or assistance of the user in the execution of the technical task of setting up and/or using of the laser device via a guided man-machine interaction, involving the determination of an energy parameter which combines an energy level and a pulse duration for the light emission by the endourological laser surgery device and the display of the first user interface object representative of the energy parameter. This permits a simplification of the use of the laser device, and renders the use thereof less vulnerable to sub-optimum parameterization. For example, the energy parameter (which is associated with a predetermined pulse duration) is the first parameter to be defined by the electronic device disclosed. For example, a frequency, (which can be seen as representing speed), can only be specified if the energy parameter has been defined.

The electronic device comprises the display, which is configured to display a user interface. A user interface comprises one or more user interface objects. A user interface may be described as a user interface screen.

The term user interface object disclosed herein refers to a graphic representation of an object which is represented on the display of the electronic device. The user interface object may be interactive with the user, or may be selectable via a user input. For example, an image (for example an icon), a button and a text (for example a hyperlink) may each optionally constitute a user interface object. The user interface object may form part of a widget. A widget may be considered as a mini-application, which may be employed by the user and generated by the user. A user interface object may comprise an invitation, an icon for launching the application and/or an action menu. An input, such as a first input and/or a second input, may comprise a touch contact (for example, a tap, a hold-down contact, or sustained pressure) and/or a contact motion (for example, a sweeping movement, e.g. for the execution of a switchover) and/or a selection of a user interface object. The contact motion may be detected by a tactile surface, for example on a screen of an electronic device. Accordingly, the display may be a touchscreen display. An input, such as a first input and/or a second input, may comprise the selection of a user interface object via a mouse or another input device. An input, such as a first input and/or a second input, may comprise a touch contact and a movement, followed by a release.

The display of the electronic device may be configured to detect a touch contact (for example, the display is a touchscreen display), wherein the input comprises a contact with the touchscreen. A touchscreen constitutes an input interface and an output interface between the electronic device and a user.

A processor of the electronic device may be configured to receive and/or transmit electrical signals from/to a display, for example a touchscreen. A display, for example a touchscreen, is configured to display a visual output to the user. The visual output optionally comprises graphics, text, icons, video, or any combination of the above (described collectively as “graphics”). For example, all or part of the visual output may be considered to constitute user interface objects.

The processor of the electronic device may be configured to display, on the display, one or more user interfaces, such as user interface screens, comprising a first user interface and/or a second user interface. A user interface may comprise one or more, for example a plurality of user interface objects. For example, the first user interface may comprise a first primary user interface object and/or a first secondary user interface object. A second user interface may comprise a second primary user interface object and/or a second secondary user interface object. A user interface object, such as the first user interface object, may represent the energy parameter disclosed herein.

A configuration scheme may comprise one or more configuration parameters, including, for example, initial configuration parameters (e.g. default parameters). In one or more examples, the configuration scheme disclosed herein is for initialization of the configuration parameter(s). The configuration scheme permits the provision of a configuration which results in the determination (and, for example, monitoring) of the energy parameter associated with the light emission by the endourological laser surgery device, wherein the energy parameter is determined based on an energy level and a pulse duration for the light emission by the endourological laser surgery device. The configuration scheme is not any of the one or more modes disclosed here such as for Coagulation, Section, Ablation, and/or Prostate.

In one or more exemplary electronic devices, the first user interface object representative of the energy parameter comprises a first primary user interface object which is representative of an energy parameter range. For example, the energy parameter range may be considered in the form of a spectrum or scale for the energy parameter. For example, the energy parameter range may comprise gradations of the energy parameter. Each gradation may be represented by a user interface object which forms part of the first primary user interface object which is representative of an energy parameter range.

In one or more exemplary electronic devices, the electronic device is configured to receive, via the input device, a user input which is indicative of the selection of an operational environment. The user input indicative of the selection of an operating environment may be executed in the form of the selection of a user interface object representative of an operational environment, which is represented on the display. The operational environment constitutes the environment in which the endourological laser surgery device is to operate and/or to be used. The operational environment thus delivers the appropriate energy parameter for the endourological area which is to receive the light application, including the following preconfigured environments: “renal <1000 HU”, “renal >1000 HU”, “ureter <1000 HU”, “ureter >1000 HU”, “tissues”. It is envisaged that an operational environment may be provided which is described as “free”, permitting the unrestricted parameterization of all parameters, with no assistance. The “free” environment is not considered as a preconfigured environment. The “tissues” operational environment corresponds to an appropriate environment for the application on endourological tissues. In one or more exemplary electronic devices, the “tissues” operational environment also generates the display of one or more other operational environments, including Prostate, Coagulation, Section (incision), and/or Excision (ablation).

It is also envisaged, for example, that the following environments may be delivered: RENAL Calculus (CaR), Urethral Calculus (CaU), Tumour (Tu), Prostate (P).

In one or more exemplary electronic devices, the electronic device is configured, in response to the reception of a user input, to display, on the display, the first primary user interface object which is representative of the energy parameter range which corresponds to the operational environment.

The operational environment also generates the display of one or more modes of use, including Coagulation, Section, Ablation, and/or Prostate. The operational environment also generates the display of one or more modes of use, for example the following, in the case of a “renal” environment: a fragmentation mode, a pulverization mode and/or a popcorning mode; for a tissue environment: an upper tract urothelial carcinoma (UTUC) mode, a section mode, a coagulation mode and/or a prostate mode. For example, the Ablation mode is the UTUC mode. For example, the term “Prostate” may be applied equally to an environment (for example the prostate) and to a mode, wherein the term “ThuFlep” (Thulium Fibre Laser Enucleation of the Prostate) can be applied for the mode of the prostate, or Enucleation.

In one or more exemplary electronic devices, the electronic device is configured to receive, via the input device, a user input which is indicative of the selection of a mode of use.

In one or more exemplary electronic devices, the electronic device is configured to determine, based on the operational environment (and, optionally, based on a selected mode), a first zone indicative of effective working and a second zone indicative of optimization. In one or more exemplary electronic devices, the first user interface object comprises a first secondary user interface object representative of the first zone, and a first tertiary user interface object representative of the second zone.

For example, by the selection of a mode, the electronic device is configured to determine a zone indicative of effective working (or an effective working zone, for example between 0 and 6 Joules), and a zone indicative of optimization (or optimization zone), which will remain within a safe zone. The optimization zone is a zone in which the desired effect is close to ideal. The zone indicative of effective working and the zone indicative of optimization fall within a safe zone, in which the user or the urologist can use the laser device with no risk. These zones may be defined by clinical studies and laboratory research works. At any time, the user may deactivate this mode, and assume a free mode. For example, in a “Urethral Calculus” environment, with a 150 micron branched fibre, the energy parameter scale may range from 0 to 2 Joules, with 0.1 J gradations, with a working zone of 0 to 1.5 J and an optimization zone of 1.5 to 2 J.

In one or more exemplary electronic devices, the electronic device is configured to display, on the display, a second user interface object which is representative of a frequency parameter for the light emission. The frequency parameter may be defined as a parameter which is indicative of the number of pulses per unit of time, for example per second, per millisecond or per microsecond. The frequency parameter may be expressed in Hertz, or Hz. The frequency parameter may be linked to the energy parameter. For example, if excessively high energy is selected, a maximum frequency level will be automatically set by the laser system. In other words, it is not possible to use a laser device at maximum energy and maximum frequency—an element of balance needs to be observed. The use of a self-limiting system might be envisaged.

In one or more exemplary electronic devices, the second user interface object comprises a second primary user interface object, which is representative of a frequency parameter range.

In one or more exemplary electronic devices, the electronic device is configured for the achievement, during the operation of the endourological laser surgery device, of a given service energy level and a given service pulse duration. In one or more exemplary electronic devices, the electronic device is configured for the determination of an energy indicator which is based upon the service energy level and the service pulse duration (for example, with respect to service executed in real time). In one or more exemplary electronic devices, the electronic device is configured for the representation, on the display, of a third user interface object, which is representative of the energy indicator. In other words, the energy indicator indicates the energy used at a given service time instant t, by the indication thereof on the energy parameter range.

In one or more exemplary electronic devices, the electronic device is configured for the representation, on the display, of a fourth user interface object, which is representative of the operational environment.

In one or more exemplary electronic devices, the electronic device is configured for the determination of a first measurement indicative of the total energy emitted by the endourological laser surgery device during the use thereof. In one or more exemplary electronic devices, the electronic device is configured for the representation, on the display, of a fifth user interface object, which is representative of the first measurement. For example, the electronic device is capable of logging the total energy delivered by the execution of an automatic calculation at the end of an application of the laser: J or J/sec or sec/J. For example, a fifth user interface object, which is representative of the first indicative measurement of total energy emitted may be displayed in real time during service. This permits a user to adjust their use of the laser device, for example to change modes by returning, for example, to a user interface which displays one or more user interface objects which are representative of operational environments.

In one or more exemplary electronic devices, the electronic device is configured for the determination of a second measurement indicative of the actual duration of the light emission by the endourological laser surgery device during the use thereof. In one or more exemplary electronic devices, the electronic device is configured for the representation, on the display, of a sixth user interface object which is representative of the second measurement. For example, the electronic device is capable of measuring the actual laser activation time and the total laser time (such as actual time and pauses) and of displaying the sixth user interface object, together with another user interface object which is representative of the total laser time, for example in the form of two needles operating on the same dial.

In one or more exemplary electronic devices, the electronic device is configured for the determination, as a function of the energy level and the frequency parameter, of a third measurement indicative of the power of light emitted by the endourological laser surgery device during the use thereof. In one or more exemplary electronic devices, the electronic device is configured for the representation, on the display, of a seventh user interface object, which is representative of the third measurement. For example, power may be calculated by multiplying energy by frequency. For example, the seventh user interface object may be expressed in numerical form, for example “27 W”.

The endourological laser surgery system may comprise a first pedal, which permits a transition from one environment to another, for example the right-hand pedal of the laser device, which can be employed as a selector. The endourological laser surgery system may comprise a second pedal, which actuates the laser device, for example the left-hand pedal. The inverse arrangement is also conceivable. A constant mode may be considered as a “cruise control”. Each pedal may be equipped with a light source, which is illuminated when the laser device is enabled or active, and extinguished when the laser device is disabled or inactive. It is envisaged that a push button can be provided between the two right- and left-hand pedals, which permits the activation (in the ON or READY position) or deactivation (in the OFF or Standby position) of the laser device.

The electronic device can be configured to emit an audio signal, e.g. with a fixed duration when the electronic device runs in Ready mode and the footswitch is pressed. During the operation of the electronic device, the emitted audio signal can vary according to the frequency of laser pulses. The electronic device can be configured to tune the audio signal level associated to each pedal (LEFT and RIGHT).

In one or more exemplary electronic devices, the electronic device is configured for the delivery of an end-of-treatment report (e.g. a review, an end of application report) for the operation executed, comprising the total energy delivered, and/or the total treatment time, and/or total application time and/or the total laser time and active time, and/or Joules/sec and sec/Joules.

In one or more example, the electronic device is configured to display a user interface object which permits the user to access one or more reports. A report may include procedure information (such as date, time, case number, environment, fibre size, fibre type, active time, total laser time, total number of pulse, and/or total energy delivered) and efficiency metrics (such as input value of the stone(s) volume, ablation efficiency and energy delivered per unit of volume).

In one or more example, the electronic device is configured to display a user interface object representative of the report, such as efficiency metrics illustrated in graph(s).

A further object of the present disclosure is a method for the operation of an endourological laser surgery system. The endourological laser surgery system comprises an endourological laser surgery device. The method comprises the acquisition of a configuration scheme of the endourological laser surgery device. The method comprises the determination, based on the configuration scheme, of an energy parameter associated with a light emission by the endourological laser surgery device. The energy parameter is determined based on an energy level and a pulse duration of the light emission by the endourological laser surgery device. The method comprises the representation, on the display, of a first user interface object which is representative of the energy parameter.

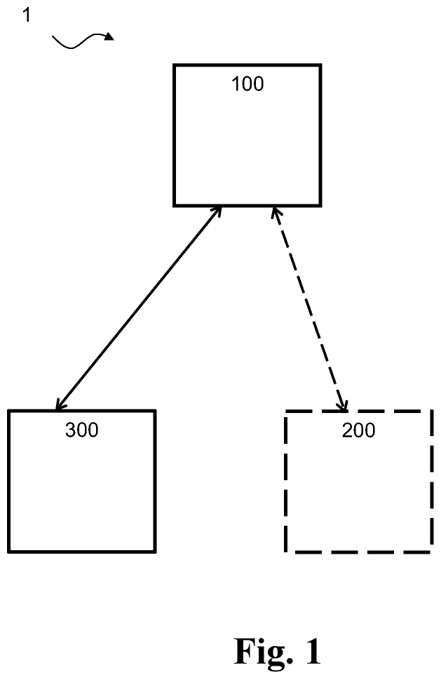

Understanding of the invention will be clarified by the following description, which is based upon potential modes of embodiment, which are explained by way of illustration and not by way of limitation, with reference to FIG. 1, which schematically outlines a potential type of architecture.

FIG. 1 is a diagram illustrating an exemplary endourological laser surgery system 1 according to the present disclosure. The endourological laser surgery system 1 comprises an endourological laser surgery device 100 and, optionally, a pedal device 200. Accordingly, the object of the invention is an electronic device 300 for the operation of an endourological laser surgery system 1.

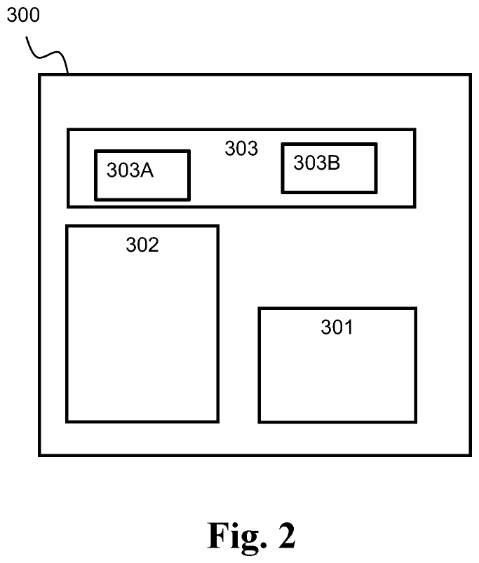

FIG. 2 is a diagram illustrating an exemplary electronic device 300 for the operation of an endourological laser surgery system according to the present disclosure.

The electronic device 300 comprises a processor 302; memory 301; an interface 303 comprising a display 303A and an input device 303B.

The electronic device 300 is configured to obtain (for example via the memory and/or the processor 302) a configuration scheme of the endourological laser surgery device.

The electronic device 300 is configured to determine (for example via the processor 302), based on the configuration scheme, an energy parameter associated with a light emission by the endourological laser surgery device. The energy parameter is determined based on an energy level and a pulse duration of the light emission by the endourological laser surgery device.

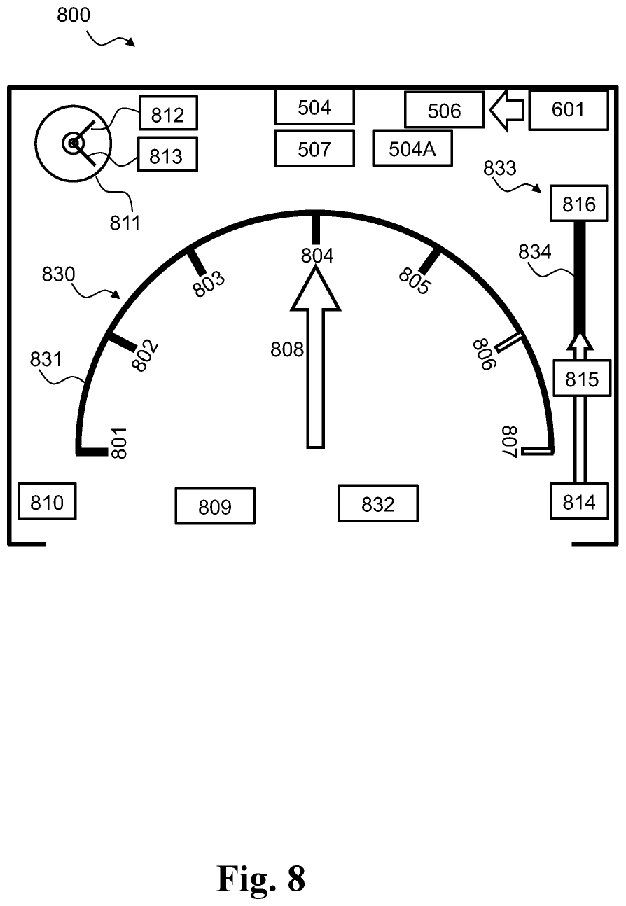

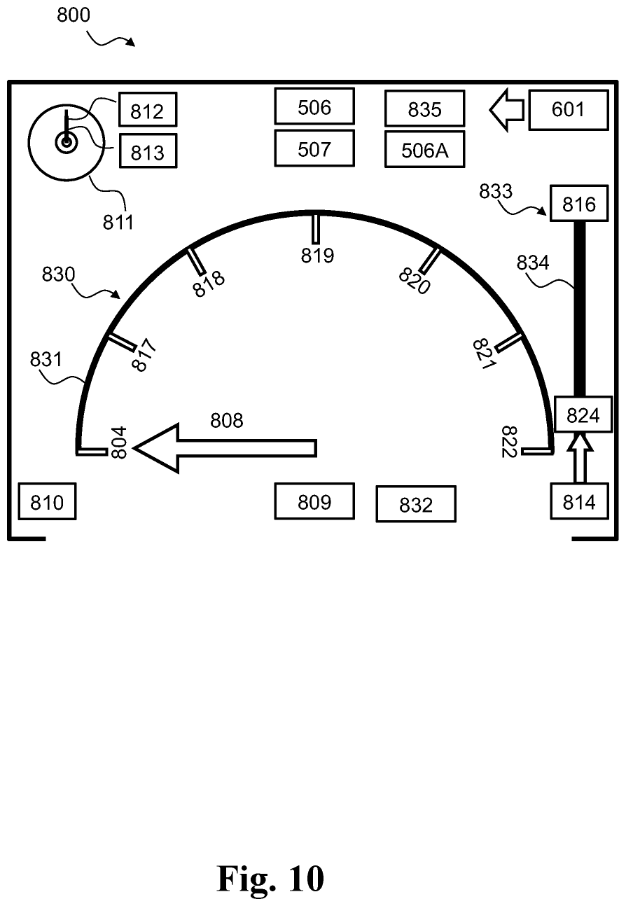

The electronic device 300 is configured to display, on the display 303A, a first user interface object which is representative of the energy parameter (for example, illustrated in FIGS. 8 and 10).

In one or more exemplary electronic devices, the first user interface object representative of the energy parameter comprises a first primary user interface object which is representative of an energy parameter range.

In one or more exemplary electronic devices, the electronic device 300 is configured to receive, via the input device 303B, a user input which is indicative of the selection of an operational environment.

In one or more exemplary electronic devices, the electronic device 300 is configured, in response to the reception of the user input, to display on the display 303A, the first representative primary user interface object for the energy parameter range which corresponds to the operational environment.

In one or more exemplary electronic devices, the electronic device 300 is configured to determine (for example, via the processor 302), based on the operational environment, a first zone indicative of effective working and a second zone indicative of optimization. In one or more exemplary electronic devices, the first user interface object comprises a first secondary user interface object representative of the first zone, and a first tertiary user interface object representative of the second zone.

In one or more exemplary electronic devices, the electronic device 300 is configured to display, on the display 303A, a second user interface object which is representative of a frequency parameter of the light emission (which may correspond, for example, to the operational mode).

In one or more exemplary electronic devices, the second user interface object comprises a second primary user interface object which is representative of a frequency parameter range.

In one or more exemplary electronic devices, the electronic device 300 is configured, during the operation of the endourological laser surgery device (such as the laser device 100 illustrated in FIG. 1) to obtain (for example via the processor 302) an energy level of the operation and a pulse duration of the operation. In one or more exemplary electronic devices, the electronic device 300 is configured to determine (for example via the processor 302) an energy indicator based upon the energy level of the operation and the pulse duration of the operation. In one or more exemplary electronic devices, the electronic device 300 is configured to display, on the display 303A, a third user interface object which is representative of the energy indicator.

In one or more exemplary electronic devices, the electronic device 300 is configured to display, on the display 303A, a fourth user interface object which is representative of the operational environment.

In one or more exemplary electronic devices, the electronic device 300 is configured to determine (for example via the processor 302) a first measurement indicative of the total energy emitted by the endourological laser surgery device during the use thereof. In one or more exemplary electronic devices, the electronic device 300 is configured to display, on the display 303A, a fifth user interface object which is representative of the first measurement.

In one or more exemplary electronic devices, the electronic device 300 is configured to determine (for example via the processor 302) a second measurement indicative of the actual duration of the light emission by the endourological laser surgery device during the use thereof. In one or more exemplary electronic devices, the electronic device 300 is configured to display, on the display 303A, a sixth user interface object which is representative of the second measurement.

In one or more exemplary electronic devices, the electronic device 300 is configured to determine (for example via the processor 302), as a function of the energy level and the frequency parameter, a third measurement indicative of the power of light emitted by the endourological laser surgery device during the use thereof. In one or more exemplary electronic devices, the electronic device 300 is configured to display, on the display 303A, a seventh user interface object with is representative of the third measurement.

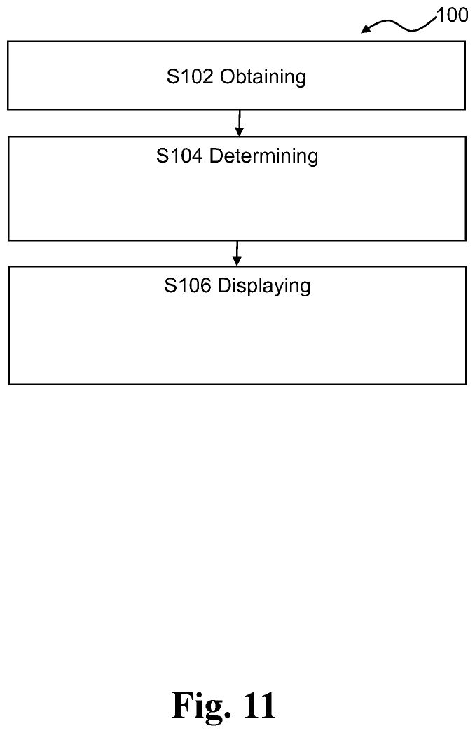

The processor circuit 302 is optionally configured to execute any of the operations described in FIG. 11 (for example, one or more of the operations S102, S104, S106). Operations on the network node 300 may be executed in the form of executable logic routines (for example, lines of code, software programs, etc.) which are saved on a non-transitory computer-readable medium (for example, memory circuits 301) and are executed by the processor circuits 302).

Moreover, operations on the network node 300 may be considered as a method which the network node 300 is configured to execute. Likewise, although the functions and operations described may be implemented in a software, a functionality of this type can also be executed via a dedicated hardware or firmware, or via a certain combination of hardware, firmware and/or software.

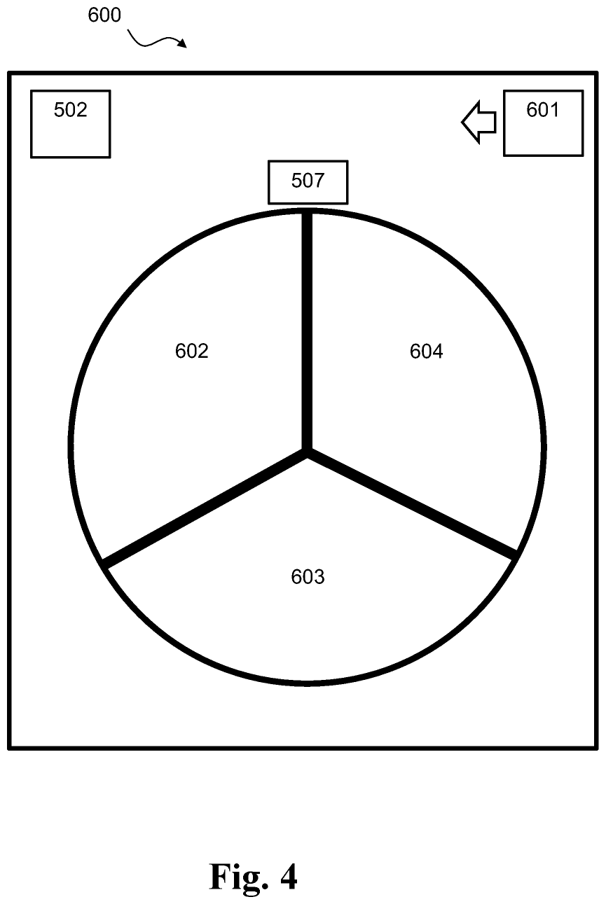

The memory 301 may be one or more of the following: a buffer memory, a flash memory, a hard disk, a removable storage medium, a non-volatile memory, a random access memory (RAM) or another appropriate device. In a typical arrangement, the memory 301 may comprise a non-volatile memory for the long-term storage of data, and a non-volatile memory which functions as a system memory for the processor 302. The memory 301 may exchange data with the processor 302 via a data bus. Control lines and an address bus between the circuits of the memory 301 and the circuits of the processor 302 may also be present (not represented in FIG. 4). The memory 301 is considered as a non-transitory computer-readable medium.

The memory 301 may be configured for the storage of configuration schemes in one part of the memory.

In one or more exemplary electronic devices, the electronic device is configured to store the selection of the operational environment by the user, such as one or more parameters may be stored in pre-settings for a given user (such as one or more of: a user profile, an environment, a location, a name, an energy parameter, a frequency parameter, a power parameter, a fibre type, and a pulse duration).

In one or more exemplary electronic devices, the electronic device is configured to reset, such as reinitialize, one or more counters, such as actual laser activation time and total laser time. The reset may occur when the electronic device determines that a new fibre is detected.

In one or more exemplary electronic devices, the electronic device is configured to generate and provide a report including one or more measurements disclosed herein, and/or one or more counters disclosed herein.

In one or more exemplary electronic devices, the electronic device is configured to generate one or more efficiency metrics indicative of an efficiency of energy delivered on a volume of tissue. For example, for a stone having a volume given as a volume input (and/or one or multiple volumes), the electronic device is configured to calculate an efficiency metric indicative of ablation efficiency (e.g. volume divided by Time in laser ON, e.g. volume/time unit). For example, for a stone having a volume given as a volume input (and/or one or multiple volumes), the electronic device is configured to calculate the energy delivered on the indicated volume of stones (such as Energy total divided by volume, e.g. volume/per energy unit).

In one or more exemplary electronic devices, the electronic device is configured to display a user interface object representative of the volume input, and to receive user input via the user interface object.

In one or more exemplary electronic devices, the electronic device is configured to include one or more volume parts. In one or more exemplary electronic devices, the electronic device is configured to display a user interface object enabling to receive a user input for adding a volume part.

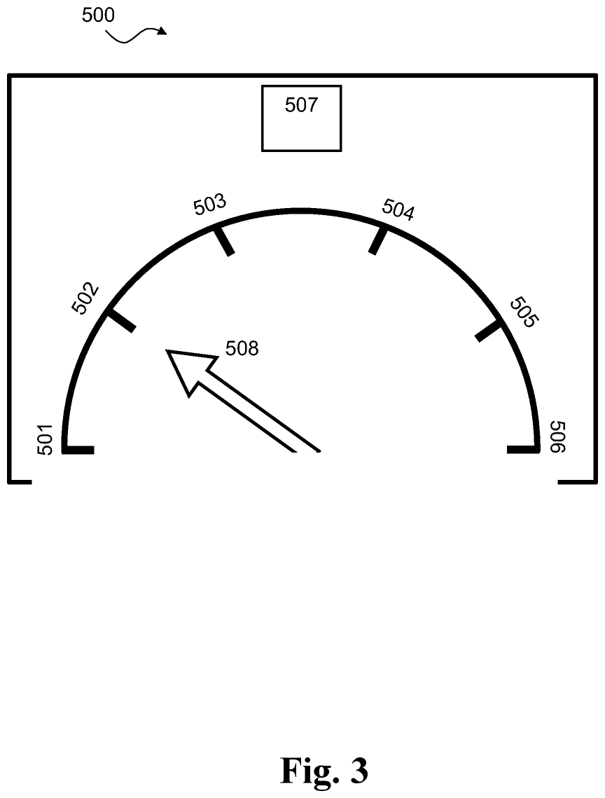

FIG. 3 shows a diagram illustrating an exemplary user interface screen 500 for the display of exemplary user interface objects which are representative of operational environments according to the present disclosure.

The user interface screen 500 comprises a user interface object 501 which is representative of a first operational environment, for example for a stone, for example the “renal <1000 HU” environment and/or a soft stone environment. “Hounsfield” denotes a Hounsfield unit, or HU.

The user interface screen 500 comprises a user interface object 502 which is representative of a second operational environment, for example for a stone, for example the “renal >1000 HU” environment, and/or a hard stone environment.

The user interface screen 500 optionally comprises a user interface object 503 which is representative of a third operational environment, for example for Benign Prostatic Hyperplasia, for example the “ureter <1000 HU” environment, and/or ureter soft stone environment.

The user interface screen 500 optionally comprises a user interface object 504 which is representative of a fourth operational environment, for example the “ureter >1000 HU” environment, and/or ureter hard stone environment.

The user interface screen 500 optionally comprises a user interface object 505 which is representative of a fifth operational environment, for example the “tissues” environment.

The user interface screen 500 optionally comprises a user interface object 506 which is representative of a sixth operational environment, for example the “free” environment.

The user interface screen 500 optionally comprises a user interface object 507 which is representative of the type of fibre employed, for example 150 microns, and/or if no fibre is connected

The user interface screen 500 optionally comprises a user interface object which is representative of an operational environment, such as for Benign Prostatic Hyperplasia (BPH).

The user interface screen 500 optionally comprises a user interface object which is representative of individual pre-settings.

The user interface screen 500 optionally comprises a user interface object which is representative of fibre guide for guiding the user in selecting the fibre, based on fibre size, and/or considering for a stone, a tissue and/or BPH.

The user interface screen 500 optionally comprises a user interface object which is representative of setting parameters of the electronic device.

The user interface screen 500 optionally comprises a user interface object which leads to a home screen when selected by a user input.

The user interface screen 500 may be displayed on the display of the electronic device disclosed, for example based on the configuration scheme of the endourological laser surgery device, for example in conjunction with an activation of the endourological laser surgery device and/or of the electronic device disclosed.

The user interface screen 500 may be displayed on the display of the electronic device disclosed, for example in order to change the operational environment.

In one or more exemplary electronic devices, the electronic device is configured to receive, via the input device, a user input indicative of the selection of an operational environment. The user input indicative of the selection of an operational environment may be executed by the selection of a user interface object (such as 501, 502, 503, 504, 505 or 506) which is representative of an operational environment which is displayed on the display. The operational environment describes the environment in which the endourological laser surgery device is to operate. The operational environment thus describes the appropriate energy parameter for the endourological area for application of the light, such as “renal <1000 HU”, “renal >1000 HU”, “ureter <1000 HU”, “ureter >1000 HU” or “tissues”. It is envisaged that an operational environment described as “free” can be provided, which permits the free parameterization of all parameters, with no assistance.

In one or more exemplary electronic devices, the electronic device is configured to store the selection of the operational environment by the user, such as one or more energy parameters may be stored in pre-settings for a given user.

In one or more exemplary electronic devices, the electronic device is configured to reset, such as reinitialize, one or more counters, such as actual laser activation time and total laser time.

The user interface screen 500 comprises a user interface object 508 which permits the selection of the operational environment. For example, the object 508 indicates the selection of the “stone” (such as “renal >1000 HU”) environment, represented by 502. After the selection of the “stone” (such as “renal >1000 HU”) environment, represented by 502, the electronic device can optionally display the user interface screen 600 illustrated in FIG. 4.

The user interface screen 500 may display the various interface objects with various shapes and in various arrangements.

FIG. 4 is a diagram illustrating an exemplary user interface screen 600 which displays exemplary user interface objects which are representative of modes associated with a selected environment according to the present disclosure.

For example, where a stone environment (such as the “renal >1000 HU” environment, such as a kidney hard stone environment) represented by 502 in FIG. 3 is selected by a user, the electronic device may optionally display the user interface screen 600.

The user interface screen 600 comprises a user interface object 602 which is representative of a first mode, for example the dusting, pulverization, or dust mode.

The user interface screen 600 optionally comprises a user interface object 603 which is representative of a second mode, for example the popcorning mode.

The user interface screen 600 optionally comprises a user interface object 604 which is representative of a third mode, for example the fragmentation mode.

The user interface screen 600 optionally comprises a user interface object 507 which is representative of the type of fibre employed, for example 150 microns.

The user interface screen 600 optionally comprises an object 601 which permits a return to a screen showing a main menu. The main menu permits the selection of operational environments. The user interface screen 600 optionally comprises an object represented in the form of an arrow, which permits a return to a preceding screen.

The user interface screen 600 optionally comprises a user interface object 502 which is representative of the operational environment selected, for example a kidney hard stone environment, such as the “renal >1000 HU” environment.

For example, where a BPH environment represented by user interface object is selected by a user, the electronic device may optionally display the user interface screen 600, wherein a user interface object is representative of an enucleation mode, and a user interface object which is representative of a vaporization mode. In the BPH mode, the user interface can lead the user to use certain fibers by requesting to select certain fibers (such as 365 microns or 550 microns) to access BPH pre-settings.

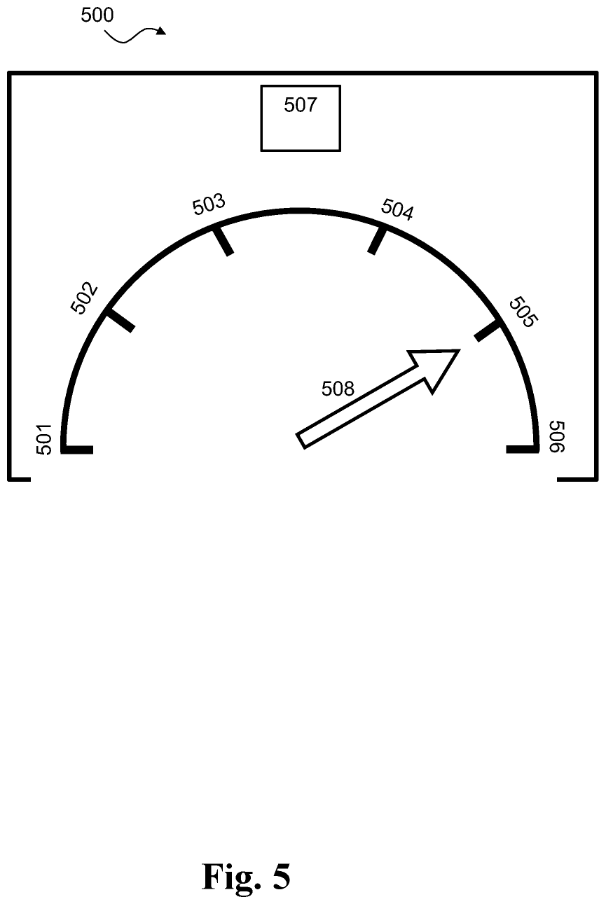

FIG. 5 is a diagram illustrating an exemplary user interface screen 500 for the display of exemplary user interface objects which are representative of operational environments according to the present disclosure.

The user interface screen 500 comprises a user interface object 501 which is representative of a first operational environment, for example the “renal <1000 HU” environment. The user interface screen 500 optionally comprises a user interface object 502 which is representative of a second operational environment, for example the “renal >1000 HU” environment. The user interface screen 500 optionally comprises a user interface object 503 which is representative of a third operational environment, for example the “ureter <1000 HU” environment. The user interface screen 500 optionally comprises a user interface object 504 which is representative of a fourth operational environment, for example the “ureter >1000 HU” environment. The user interface screen 500 comprises a user interface object 505 which is representative of a fifth operational environment, for example the “tissues” environment. The user interface screen 500 optionally comprises a user interface object 506 which is representative of a sixth operational environment, for example the “free” environment.

The user interface screen 500 optionally comprises a user interface object 507 which is representative of the type of fibre employed, for example 150 microns, or 200 microns, or 270 microns, or 365 microns, or 550 microns, or 800 microns.

It is envisaged that the representative user interface object 507 for the type of fibre employed can appear on all screens, immediately the laser fibre is connected to the laser machine. This connection is possible at any time and, accordingly, it may be possible for the icon to be displayed in consequence. For example, when no fiber or a non-recommended fiber is connected, a warning can be displayed by the electronic device.

Optionally, the operational environment also generates the display of one or more modes of use, for example for a renal environment: a fragmentation mode, a pulverization mode and/or a popcorning mode; for a tissue environment: an upper tract urothelial carcinoma (UTUC) mode, a section mode, a coagulation mode and/or a prostate mode. Optionally, the operational environment also generates the display of one or more modes of use, for a tissue environment: an ablation mode, a section mode, a coagulation mode and/or an incision mode.

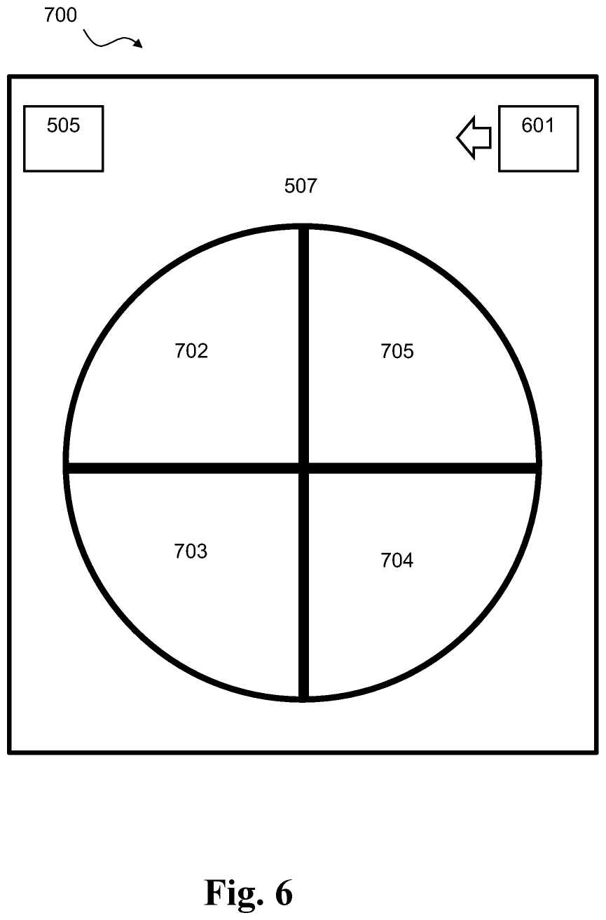

The user interface screen 500 comprises a user interface object 508 which permits the selection of an operational environment. For example, the object 508 indicates the selection of the “tissue” environment, represented by 505. Once the “tissues” environment, represented by 505, has been selected, the electronic device can optionally display the user interface screen 700 illustrated in FIG. 6 with one or more modes of use, for a tissue environment: an ablation mode, a section mode, a coagulation mode and/or an incision mode.

FIG. 6 is a diagram illustrating an exemplary user interface screen 700 for the display of exemplary user interface objects which are representative of modes associated with a selected environment according to the present disclosure.

For example, where the “tissues” environment, represented by 505 in FIG. 5, is selected by a user, the electronic device may optionally display the user interface screen 700.

The user interface screen 700 comprises a user interface object 702 which is representative of a mode, for example the UTUC mode.

The user interface screen 700 optionally comprises a user interface object 703 which is representative of a mode, for example the coagulation mode.

The user interface screen 700 optionally comprises a user interface object 704 which is representative of a mode, for example the prostate mode.

The user interface screen 700 optionally comprises a user interface object 705 which is representative of a mode, for example the section mode.

The user interface screen 700 optionally comprises a user interface object 507 which is representative of the type of fibre employed, for example 150 microns.

The user interface screen 700 optionally comprises a user interface object 505 which is representative of the operational environment selected, for example the “tissues” environment.

The user interface screen 700 optionally comprises an object which permits a return to a screen showing a main menu for the selection of environments. The user interface screen 700 optionally comprises an object represented in the form of an arrow which permits a return to a preceding screen.

For example, where a tissue environment represented by user interface object is selected by a user, the electronic device may optionally display the user interface screen 700, wherein a user interface object is representative of an ablation mode, and a user interface object 703 which is representative of a coagulation mode, and user interface 705 object representative of an incision mode.

Any of the user interface screen disclosed herein may optionally include a user interface object representative of a pedal for activation and/or an activated pedal.

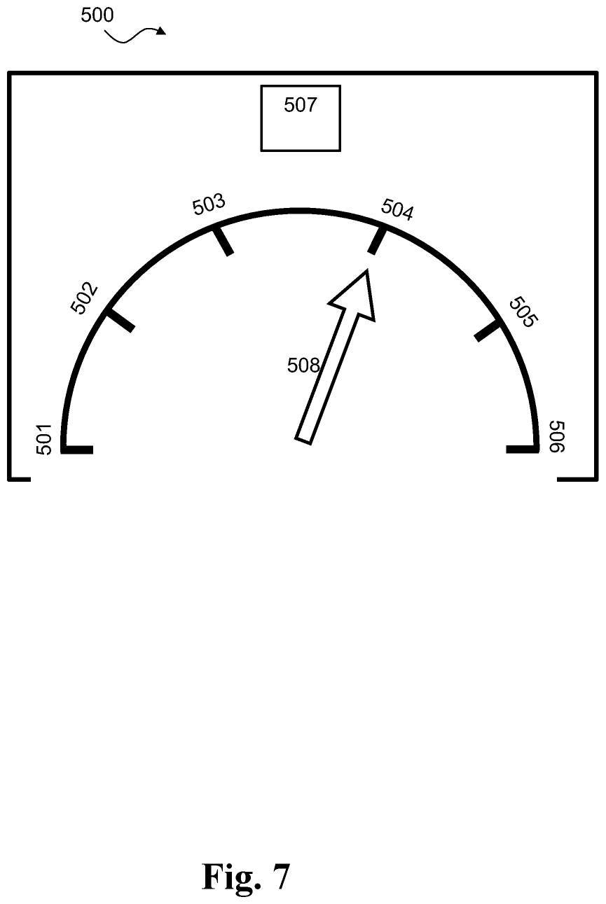

FIG. 7 is a diagram illustrating an exemplary user interface screen 500 for the display of exemplary user interface objects which are representative of operational environments according to the present disclosure.

The user interface screen 500 comprises a user interface object 501 which is representative of a first operational environment, for example the “renal <1000 HU” environment. The user interface screen 500 optionally comprises a user interface object 502 which is representative of a second operational environment, for example the “renal >1000 HU” environment. The user interface screen 500 optionally comprises a user interface object 503 which is representative of a third operational environment, for example the “ureter <1000 HU” environment. The user interface screen 500 comprises a user interface object 504 which is representative of a fourth operational environment, for example the “ureter >1000 HU” environment. The user interface screen 500 optionally comprises a user interface object 505 which is representative of a fifth operational environment, for example the “tissues” environment. The user interface screen 500 optionally comprises a user interface object 506 which is representative of a sixth operational environment, for example the “free” environment.

The user interface screen 500 optionally comprises a user interface object 507 which is representative of the type of fibre employed, for example 150 microns.

The user interface screen 500 comprises a user interface object 508 (for example, for the representation of selection) for the selection of the operational environment. For example, the object 508 indicates the selection of the “ureter >1000 HU” environment, represented by 504. Following the selection of the “ureter >1000 HU” environment, represented by 504, the electronic device can optionally display the user interface screen 800 illustrated in FIG. 8.

FIG. 8 is a diagram illustrating an exemplary user interface screen 800 for the display, inter alia, of a first user interface object which is representative of the energy parameter according to the present disclosure.

The user interface screen 800 comprises a first user interface object 830 which is representative of the energy parameter according to the present disclosure. The energy parameter is associated with a light emission by the endourological laser surgery device. The energy parameter is determined based on an energy level and a pulse duration for the light emission by the endourological laser surgery device, for example for a selected environment.

In one or more exemplary electronic devices, the first user interface object representative of the energy parameter comprises a first primary user interface object 831 which is representative of an energy parameter range. For example, the energy parameter range may be considered as a spectrum or scale for the energy parameter. For example, the energy parameter range comprises gradations of the energy parameter. Each gradation may be represented by a user interface object (such as 801, 802, 803, 804, 805, 806 or 807) which forms part of the first representative primary user interface object for the energy parameter range. For example, the range for the “ureter >1000 HU” environment may range from 0.1 J to 0.4 J, in gradations of 0.05 (for example, 801 representing 0.1 J, 802 representing 0.15 J, 803 representing 0.2 J, 804 representing 0.25 J, 805 representing 0.3 J, 806 representing 0.35 J, and 807 representing 0.4 J). These values are illustrative examples—other values may be applied. The energy parameter range under use may be displayed on the side of the dial. The user interface can include a user interface object (such as a “+” and “−” icons) for enabling user input to increase and decrease the energy parameter.

In one or more exemplary electronic devices, the electronic device is configured, in receive a user input on any of UI object 801, 802, 803, 804, 805, 806, 807 and any gradation therefore to select a value for the energy parameter. For example, when the increase in the energy parameter leads to an exceeding of the recommended energy parameter and/or an exceeding of the power limit, the electronic device may be configured to display a pop-up message to indicate the exceeding and inquiring about switching to free mode.

In one or more exemplary electronic devices, the electronic device is configured, in response to the reception of the user input, to display, on the display, the first representative primary user interface object 831 for the energy parameter range corresponding to the operational environment.

In one or more exemplary electronic devices, the electronic device is configured to receive, via the input device, a user input which is indicative of the selection of a mode of use, for example by the selection of a screen object 500 illustrated in FIGS. 3, 5, and/or 7.

In one or more exemplary electronic devices, the electronic device is configured to determine, based on the operational environment (and, optionally, based on a selected mode), a first zone indicative of effective working and a second zone indicative of optimization. In one or more exemplary electronic devices, the first user interface object 830 comprises a first secondary user interface object which is representative of the first zone, and a first tertiary user interface object which is representative of the second zone. The first secondary user interface object which is representative of the first zone is illustrated on the screen 800 by the black gradations 801, 802, 803, 804, 805. The first tertiary user interface object which is representative of the second zone is illustrated on the screen 800 by the white gradations 806, 807.

In one or more exemplary electronic devices, the electronic device is configured, during the operation of the endourological laser surgery device, to obtain a service energy level and a service pulse duration. In one or more exemplary electronic devices, the electronic device is configured to determine an energy indicator based upon the service energy level and the service pulse duration (for example, with respect to service executed in real time). In one or more exemplary electronic devices, the electronic device is configured to display, on the display, a third user interface object 808 which is representative of the energy indicator. In other words, the energy indicator indicates the energy used at a given service time instant t, by the indication thereof on the energy parameter range represented by 831. The user interface screen 800 optionally comprises the object 808. The object 808 may incorporate an arrow point which indicates the selection of the energy level for each laser pulse.

In one or more exemplary electronic devices, the electronic device is configured to display, on the display, a second user interface object 833 which is representative of a frequency parameter for the light emission. The frequency parameter may be defined as a parameter which is indicative of the number of pulses per unit of time, for example per second, per millisecond or per microsecond. The frequency parameter is independent of the energy parameter. The user interface screen 800 optionally comprises the object 833.

In one or more exemplary electronic devices, the second user interface object 833 comprises a second primary user interface object 834 which is representative of a frequency parameter range. In one or more exemplary electronic devices, the second user interface object 833 comprises a second secondary user interface object 815 which is representative of the service frequency at the present time instant t. In one or more exemplary electronic devices, the second user interface object 833 comprises an object 814 which is indicative of the minimum frequency and an object 816 which is indicative of the maximum frequency for this environment and/or this mode.

In one or more exemplary electronic devices, the user interface 800 can include a user interface object (such as a “+” and “−” icons) for enabling user input to increase and decrease the frequency parameter.

The frequency range can be displayed as a frequency dial with graduations represented as dots to show the available frequency range during use. Depending on the energy levels used, the range of frequency can adapt to not exceed limits on total power

A frequency parameter can be increased as e.g.:
0 to 10 or 20 Hz: 2 Hz steps
10 or 20 to 30 or 50 Hz: 5 Hz steps
50 to 100 Hz: 10 Hz steps
100 to 400 Hz: 20 Hz steps; and/or
400 to MAX Hz: 100 Hz steps

Other step values can be used. Changing the frequency value may not require pressing any confirmation icon, and can be effective immediately after changing the value and may not change the status of the laser.

For example, when the increase in the frequency parameter leads to an exceeding of the of the power limit defined for patient's safety, the electronic device may be configured to display a pop-up message to indicate the exceeding and inquiring about switching to free mode.

A power limit may be defined by the machine power limit. It cannot be possible to exceed the power limit. For example, increasing the energy or frequency value above the power limit value can lead to display that the energy will be decrease to achieve a frequency. For example, depending on the parameter being increased, the other parameter can automatically be lowered to achieve a power of 60 W (for example: when at 60 W of power, frequency decreased if energy increased).

In one or more exemplary electronic devices, the electronic device is configured to display, on the display, a fourth user interface object 504 which is representative of the operational environment and, optionally, a user interface object 504A which is representative of the mode selected. The user interface screen 800 optionally comprises the object 504 and the object 504A. The object 504A may be displayed in the form of a letter, such as D for Dusting, PC for Pop-corning, or F for Fragmentation. It is envisaged that the object 504A will permit the user to select the mode by selecting the object 504A, for example, by pressing on the object 504, it is possible to switch the mode from Dusting to Pop-corning, and then to Fragmentation, or vice versa.

In one or more exemplary electronic devices, the electronic device is configured to determine a first measurement indicative of the total energy emitted by the endourological laser surgery device during the use thereof. In one or more exemplary electronic devices, the electronic device is configured to display, on the display, a fifth user interface object 809 which is representative of the first measurement. The object 809 may represent, for example, the logging of successive Joules generated or employed by the urologist. This therefore corresponds, for example, to a cumulative energy dose at a given time instant t.

For example, the electronic device is capable of logging the total energy delivered by the execution of an automatic calculation at the end of treatment: J/sec, or J or sec/J. For example, a fifth user interface object 809 which is representative of the first measurement indicative of total energy emitted may be displayed in real time during service. This permits a user to adjust their use of the laser device, for example to change modes by returning, for example, to a user interface which displays one or more user interface objects which are representative of operational environments. The user interface screen 800 optionally comprises the object 809. The object may, when selected, deliver a treatment report indicating the total energy generated, the total laser time and the effective laser time, and J/Sec, Sec/Joules. This object might appear immediately the laser device is in the STANDBY position and/or when the laser fibre is disconnected at the end of treatment.

The screen or display may change colour upon the switchover from “ready” mode to “standby” mode, for example from green in “standby” mode to red in “ready” mode, on all or part of the screen.

In one or more exemplary electronic devices, the electronic device is configured to determine a second measurement indicative of the actual duration of the light emission by the endourological laser surgery device during the use thereof. In one or more exemplary electronic devices, the electronic device is configured to display, on the display, a sixth user interface object 812 which is representative of the second measurement. For example, the electronic device is capable of measuring the actual laser activation time and the total laser time (such as actual time and pauses) and of displaying the sixth user interface object 812, together with another user interface object 813 which is representative of the total laser time, for example in the form of two needles operating on the same dial. The user interface screen 800 optionally comprises the object 812 and/or the object 813. The user interface screen 800 optionally comprises the object 811 in the form of a clock, for the display of the object 812 and/or the object 813.

In one or more exemplary electronic devices, the electronic device is configured to determine, as a function of the energy level and the frequency parameter, a third measurement indicative of the power of light (the product of the energy parameter selected multiplied by the frequency level) emitted by the endourological laser surgery device during the use thereof. In one or more exemplary electronic devices, the electronic device is configured to display, on the display, a seventh user interface object 832 which is representative of the third measurement. For example, power may be calculated by the multiplication of energy by frequency. For example, if the energy level or frequency level changes, the total power expressed in Watts will vary accordingly: Energy (J)×Frequency (Hz)=Power expressed in Watts (W). For example, the seventh user interface object 832 may be expressed in numerical form, for example “27 W”. The user interface screen 800 optionally comprises the object 832.

The user interface screen 800 optionally comprises a user interface object 507 which is representative of the type of fibre employed, for example 150 microns.

The user interface screen 800 optionally comprises the user interface object 810 which permits the activation or otherwise of a pointer.

The user interface screen 800 optionally comprises an object 601 which permits a return to a screen showing a main menu for the selection of an environment. The user interface screen 800 optionally comprises an object represented in the form of an arrow, which permits a return to a preceding screen.

The user interface screen 800 optionally comprises the object 506 for accessing the “free” environment.

The user interface screen 800 optionally comprises the user interface object representative of beam intensity, which permits the user to adjust the beam intensity and/or to tune an aiming beam. The user interface screen 800 can include a user interface object (such as a “+” and “−” icons) for enabling user input to increase and decrease the beam intensity. For example, pressing the user interface object representative of beam intensity (such as an aiming beam icon) again or pressing any other part of the screen may save the current intensity and revert the user interface object back to its original shape.

The user interface screen 800 optionally comprises the user interface object, which permits the user to save and store the present setting as individual pre-settings.

For example, each mode can have its own pre-settings. For example, changes made within the pre-setting range can remain when scrolling between modes as long as the electronic device is not switched to the Free mode or taken back to the home screen and/or to the environment selection screen.

A colour code may be applied to the user interface screen, such as a first colour when the laser source is not activated, and the laser system cannot emit energy, a second colour when the laser source is activated, and the laser system can emit energy, and a third colour when the laser source is activated, and the laser system is emitting energy.

The user interface screen 800 optionally comprises user interface objects, which permits the user to select a pulsed mode (e.g. with a pulse duration) or a continuous wave mode. An icon can support a user selection of a pulse duration in pulsed mode, such as short, medium, or long pulse duration. The pulse duration can affect a peak power of the emitted radiation (e.g. short pulse can lead to high peak power, long pulse can lead to low peak power). While in Pulsed mode, energy and frequency can be changed. In continuous wave mode, the energy parameter, the frequency parameter, and the pulse duration are not modifiable, but the power parameter can be changed.

In a preconfigured environment or preconfigured mode (such as the ureter, renal or prostate modes illustrated), ranges for the energy parameter and the frequency parameter are fixed, and the energy parameter corresponds to a combination of the energy level in Joules and a pulse duration in units of time.

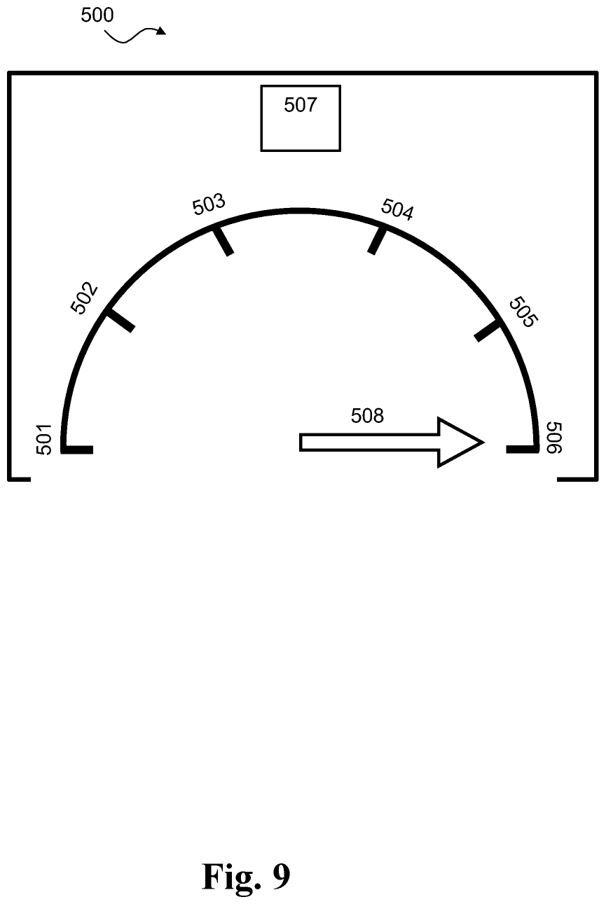

FIG. 9 is a diagram illustrating an exemplary user interface screen 500 for the display of exemplary user interface objects which are representative of operational environments according to the present disclosure.

The user interface screen 500 optionally comprises a user interface object 501 which is representative of a first operational environment, for example the “renal <1000 HU” environment. The user interface screen 500 optionally comprises a user interface object 502 which is representative of a second operational environment, for example the “renal >1000 HU” environment. The user interface screen 500 optionally comprises a user interface object 503 which is representative of a third operational environment, for example the “ureter <1000 HU” environment. The user interface screen 500 optionally comprises a user interface object 504 which is representative of a fourth operational environment, for example the “ureter >1000 HU” environment. The user interface screen 500 optionally comprises a user interface object 505 which is representative of a fifth operational environment, for example the “tissues” environment. The user interface screen 500 comprises a user interface object 506 which is representative of a sixth operational environment, for example the “free” environment.

The user interface screen 500 optionally comprises a user interface object 507 which is representative of the type of fibre employed, for example 150 microns.

The user interface screen 500 comprises a user interface object 508 (for example, representative of selection) which permits the selection of the operational environment. For example, the object 508 indicates the selection of the “free” environment represented by 506. Further to the selection of the “free” environment represented by 506, the electronic device may optionally display the user interface screen 800 illustrated in FIG. 10.

FIG. 10 is a diagram illustrating an exemplary user interface screen 800 for the “free” operational environment according to the present disclosure. The user interface screen 800 comprises a first user interface object 830 which is representative of the energy parameter according to the present disclosure. The energy parameter is associated with a light emission by the endourological laser surgery device. The energy parameter is determined based on an energy level and a pulse duration for the light emission by the endourological laser surgery device, for example for a selected environment.

In the “free” mode, when the energy parameter is selected, the pulse duration will not be programmed automatically, but can be programmed according to a deliberate preference of the operator, followed by the selection of the frequency level for the definition of the execution speed. The user interface screen 800 comprises a user interface object 835 which permits the selection of the pulse duration, such as Long, Medium, or Short (for example, Long Path LP, Medium Path MP, or Short Path SP, where S stands for Short). This only applies in the “free” mode.

In one or more exemplary electronic devices, the first user interface object representative of the energy parameter comprises a first primary user interface object 831 which is representative of an energy parameter range. For example, the energy parameter range may be considered in the form of a spectrum or scale for the energy parameter. For example, the energy parameter range comprises gradations of the energy parameter. Each gradation may be represented by a user interface object (such as 804, 817, 818, 819, 820, 821, 822) which forms part of the first primary user interface object 831 which is representative of an energy parameter range. For example, the range for the “free” environment may extend from 0.25 J to 6 J.

In one or more exemplary electronic devices, the electronic device is configured, in response to the reception of the user input, to display, on the display, the first representative primary user interface object 831 for the energy parameter range corresponding to the operational environment.

In one or more exemplary electronic devices, the electronic device is configured to receive, via the input device, a user input indicative of the selection of a mode of use, for example by the selection of an object from the screen 500 illustrated in FIGS. 3, 5, and/or 7.

The first tertiary user interface object representative of the second zone is illustrated on the screen 800 by the white gradations 804, 817, 818, 819, 820, 821, 822.

In one or more exemplary electronic devices, the electronic device is configured for the achievement, during the operation of the endourological laser surgery device, of a given service energy level and a given service pulse duration. In one or more exemplary electronic devices, the electronic device is configured to determine an energy indicator based upon the service energy level and the service pulse duration (for example, with respect to service executed in real time). In one or more exemplary electronic devices, the electronic device is configured for the representation, on the display, of a third user interface object 808, which is representative of the energy indicator. In other words, the energy indicator indicates the energy used at a given service time instant t, by the indication thereof on the energy parameter range represented by 831. The user interface screen 800 optionally comprises the object 808.

In one or more exemplary electronic devices, the electronic device is configured for the representation, on the display, of a second representative user interface object 833 for a frequency parameter for the light emission. The frequency parameter may be defined as a parameter which is indicative of the number of pulses per unit of time, for example per second, per millisecond, or per microsecond. The frequency parameter is independent of the energy parameter. The user interface screen 800 optionally comprises the object 833.

In one or more exemplary electronic devices, the second user interface object 833 comprises a second primary user interface object 834 which is representative of a frequency parameter range. In one or more exemplary electronic devices, the second user interface object 833 comprises a second secondary user interface object 824 representative of the service frequency at the present time instant t. In one or more exemplary electronic devices, the second user interface object 833 comprises an object 814 which is indicative of the minimum frequency and an object 816 which is indicative of the maximum frequency for this environment and/or this mode.

In one or more exemplary electronic devices, the electronic device is configured for the representation, on the display, of a fourth representative user interface object 506 for the FREE operational environment in FIG. 10 and, optionally, of a representative object 506A for the mode selected. The user interface screen 800 optionally comprises the object 506 and the object 506A. The object 506A may be displayed in the form of a letter or a number. It is envisaged that the object 506A will permit the user to select a mode by selecting the object 506A, for example, by pressing on the object 506, it is possible to switch from a first mode to a second mode, and thereafter to a third mode, etc.

In one or more exemplary electronic devices, the electronic device is configured for the determination of a first measurement indicative of the total energy emitted by the endourological laser surgery device during the use thereof. In one or more exemplary electronic devices, the electronic device is configured for the representation, on the display, of a fifth user interface object 809 which is representative of the first measurement. For example, the electronic device is capable of logging the total energy delivered by the execution of an automatic calculation at the end of treatment: J/sec. For example, a fifth user interface object 809, which is representative of the first measurement indicative of total energy emitted may be displayed in real time during service. This permits a user to adjust their use of the laser device, for example to change modes by returning, for example, to a user interface which displays one or more user interface objects which are representative of operational environments. The user interface screen 800 optionally comprises the object 809.

In one or more exemplary electronic devices, the electronic device is configured for the determination of a second measurement indicative of the actual duration of the light emission by the endourological laser surgery device during the use thereof. In one or more exemplary electronic devices, the electronic device is configured for the representation, on the display, of a sixth user interface object 812 which is representative of the second measurement. For example, the electronic device is capable of measuring the actual laser activation time and the total laser time (actual time and pauses) and of displaying the sixth user interface object 812, together with another user interface object 813 which is representative of the total laser time, for example in the form of two needles operating on the same dial. The user interface screen 800 optionally comprises the object 812 and/or the object 813. The user interface screen 800 optionally comprises the object 811 in the form of a clock for the display of the object 812 and/or the object 813.

In one or more exemplary electronic devices, the electronic device is configured for the determination, as a function of the energy level and the frequency parameter, of a third measurement indicative of the power of light emitted by the endourological laser surgery device during service. In one or more exemplary electronic devices, the electronic device is configured for the representation, on the display, of a seventh user interface object 832, which is representative of the third measurement. For example, power may be calculated by multiplying energy by frequency. For example, the seventh user interface object 832 may be expressed in numerical form, for example “0 W”. The user interface screen 800 optionally comprises the object 832.

The user interface screen 800 optionally comprises a user interface object 507 which is representative of the type of fibre employed, for example 150 microns.

The user interface screen 800 optionally comprises an object 810 for the activation or otherwise of a pointer.

The user interface screen 800 optionally comprises an object 601 which permits a return to a screen showing a main menu. The user interface screen 800 optionally comprises an object represented in the form of an arrow, which permits a return to a preceding screen.

The configuration scheme delivers a series of user operations in the following order, in the free mode: 1. selection of the energy level, 2. selection of the pulse duration, and 3. selection of frequency, wherein frequency is considered as a speed variator.

For example, an end of procedure is triggered by one or more of:
Machine shutdown
Fibre disconnection
By pressing the “Reset” icon.

The end of the procedure can automatically save the data and reset all counters and timers to 0.

FIG. 11 is a logic diagram illustrating an exemplary method 100 for offsetting up an endourological laser surgery system according to the present disclosure. The method 100 may be a method for monitoring an endourological laser surgery system according to the present disclosure.

The endourological laser surgery system comprises an endourological laser surgery device. The method 100 comprises obtaining S102 a configuration scheme of the endourological laser surgery device. The method 100 comprises determining, S104, based on the configuration scheme, an energy parameter associated with a light emission by the endourological laser surgery device. The energy parameter is determined based on an energy level and a pulse duration of the light emission by the endourological laser surgery device. The method comprises displaying S106, on the display, a first user interface object representative of the energy parameter.

The use of the terms “first”, “second”, “third” and “fourth”, “primary”, “secondary”, “tertiary”, etc., does not imply any particular order—these terms are included for the identification of individual elements. Moreover, the use of the terms “first”, “second”, “third” and “fourth”, “primary”, “secondary”, “tertiary”, etc. does not signify any order of importance—rather, the terms “first”, “second”, “third” and “fourth”, “primary”, “secondary”, “tertiary”, etc. are employed to distinguish one element from another. It should be observed that the terms “first”, “second”, “third” and “fourth”, “primary”, “secondary”, “tertiary”, etc. are employed here and elsewhere for tagging purposes only, and are not intended to designate any spatial or temporal order. Moreover, the tagging of a first element does not imply the presence of a second element, and vice versa.

It will be observed that FIGS. 1-11 include certain circuits or operations which are represented by a solid line, and certain circuits or operations which are illustrated by a broken line. Circuits or operations represented by a solid line are circuits or operations which are included in the widest-ranging example. Circuits or operations represented by a broken line are examples which may be included in the widest-ranging example, or are optional, or constitute further circuits or operations which may be included over and above exemplary circuits or operations which are represented by a solid line. It should be observed that these operations do not necessarily need to be executed in the order described. Moreover, it should be observed that the execution of all operations is not mandatory. Exemplary operations may be executed in any order and in any combination.

It should be observed that the term “comprising” does not necessarily exclude the presence of elements or steps other than those listed.

It should be observed that the words “a” or “an” preceding an element do not exclude the presence of a plurality of such elements.

It should further be observed that reference symbols do not limit the scope of claims, that examples may be deployed, at least in part, via both hardware and software, and that a plurality of “means”, “units” or “devices” may be represented by the same hardware.

The various exemplary methods, devices, nodes, and systems described herein are described in the general context of steps or processes according to the method, which may be deployed, according to one aspect, by a computer program product incorporated in a medium which is computer-readable only, including instructions which are executable by computer, such as program code, executed by computers in network environments. The computer-readable medium of the ONU may incorporate removable and non-removable storage peripherals, including, but not by way of limitation, read-only memory (ROM), random-access memory (RAM), compact disks (CDs), digital versatile disks (DVDs), etc. In general, program circuits may include routines, programs, objects, components, data structures, etc. which execute specific tasks or which implement specific types of abstract data. Instructions which are executable by computer, associated data structures and program circuits represent examples of program code for the execution of steps in the methods described herein. The particular sequence of such executable instructions or associated data structures is representative of exemplary corresponding acts for the deployment of functions described in said steps or methods.

Notwithstanding the presentation and description of characteristics, it will be understood that these are not intended to limit the disclosure claimed, and it will be evident to a person skilled in the art that various changes and modifications can be implemented without departing from the scope of the disclosure claimed. The description and the drawings, in consequence, are to be considered as illustrative rather than restrictive. The disclosure claimed is intended to encompass all alternatives, modifications, and equivalents.

Although the above description is based upon particular modes of embodiment, said description in no way limits the scope of the invention, and modifications may be implemented, particularly by the substitution of technical equivalents, or by a different combination of all or part of the above-mentioned characteristics.

1.-13. (canceled)

14. An endourological laser surgery system, comprising:
a laser to provide light emission for endourological surgery;
an electronic device coupled to the laser, comprising:
a processor;
an interface comprising a display and an input device, and;
memory storing instructions that when executed by the processor cause the electronic device to:
obtain a configuration scheme;
determine, based on the configuration scheme, an energy parameter associated with a light emission by the laser, wherein the energy parameter is determined based on an energy level and a pulse duration of the light emission by the laser;
display, on the display, a first user interface object representative of the energy parameter; and
control the laser based on the energy parameter.

15. The laser surgery system of claim 14, wherein the first user interface object representative of the energy parameter comprises a first primary user interface object which is representative of an energy parameter range.

16. The laser surgery system of claim 15, wherein:
the system is configured to receive, via the input device, a user input which is indicative of the selection of an operational environment; and
the instructions cause the electronic device to control the laser based on the operational environment.

17. The laser surgery system of claim 16, wherein the instructions cause the electronic device, in response to the receipt of the user input, to display, on the display, the first primary user interface object which is representative of the energy parameter range which corresponds to the operational environment.

18. The laser surgery system of claim 16, wherein:
the instructions cause the electronic device to determine, based on the operational environment, a first zone indicative of effective working and a second zone indicative of optimization; and
the first user interface object comprises a user interface object representative of the first zone, and a user interface object representative of the second zone.

19. The laser surgery system of claim 16, wherein the instructions cause the electronic device to display, on the display, a user interface object which is representative of the operational environment.

20. The laser surgery system of claim 16, wherein:
the system is configured to receive, via the input device, a user input which is indicative of the selection of a mode of use; and
the instructions cause the electronic device to control the laser based on the mode of use.

21. The laser surgery system of claim 20, wherein:
the instructions cause the electronic device to determine, based on the operational environment and the mode of use, a first zone indicative of effective working and a second zone indicative of optimization; and
the first user interface object comprises a user interface object representative of the first zone, and a user interface object representative of the second zone.

22. The laser surgery system of claim 14, wherein the instructions cause the electronic device to display, on the display, a user interface object which is representative of a frequency parameter for the light emission.

23. The laser surgery system of claim 22, wherein the instructions cause the electronic device to display, on the display, a user interface object which is representative of a frequency parameter range.

24. The laser surgery system of claim 14, wherein the instructions cause the electronic device to:
achieve a given service energy level and a given service pulse duration;
determine an energy indicator based on the service energy level and the service pulse duration; and
display, on the display, a user interface object which is representative of the energy indicator.

25. The laser surgery system of claim 14, wherein the instructions cause the electronic device to:
determine a first measurement indicative of the total energy emitted by the laser during the use thereof; and
display, on the display, a user interface object which is representative of the first measurement.

26. The laser surgery system of claim 14, wherein the instructions cause the electronic device to:
determine a second measurement indicative of the actual duration of the light emission by the laser during the use thereof; and
display, on the display, a user interface object which is representative of the second measurement.

27. The laser surgery system of claim 14, wherein the instructions cause the electronic device to:
determine, as a function of the energy level and the frequency parameter, a third measurement indicative of the power of light emitted by the laser during the use thereof; and
display, on the display, a user interface object which is representative of the third measurement.

28. An endourological laser surgery system, comprising:
a laser configured to provide light emission for endourological surgery;
an electronic device coupled to the laser, comprising:
a processor;
an interface comprising a display and an input device, and;
memory storing instructions that when executed by the processor cause the electronic device to:
obtain a configuration scheme;
receive, via the input device, a user input which is indicative of the selection of an operational environment;
receive, via the input device, a user input which is indicative of the selection of a mode of use;
determine, based on the configuration scheme, an energy parameter associated with a light emission by the laser, wherein the energy parameter is determined based on an energy level and a pulse duration of the light emission by the laser;
determine, based on the operational environment, a first zone indicative of effective working and a second zone indicative of optimization;
display, on the display, a user interface object representative of the energy parameter, a user interface object representative of the operational environment, a user interface object representative of the mode of use, a user interface object representative of the first zone, and a user interface object representative of the second zone; and
control the laser based on the energy parameter, the operational environment and the mode of use.

29. The laser surgery system of claim 28, wherein the instructions cause the electronic device to display, on the display, a user interface object which is representative of a frequency parameter for the light emission.

30. The laser surgery system of claim 29, wherein the instructions cause the electronic device to display, on the display, a user interface object which is representative of a frequency parameter range.

31. A method for operating an electronic device to control an endourological laser surgery system including a laser to provide light emission for endourological surgery, the method comprising:
obtaining, by the electronic device, a configuration scheme;
determining, by the electronic device based on the configuration scheme, an energy parameter associated with a light emission by the laser, wherein the energy parameter is determined based on an energy level and a pulse duration of the light emission by the laser;
displaying, by the electronic device on the display, a user interface object which is representative of the energy parameter; and
controlling, by the electronic device, the laser based on the energy parameter.

32. The method of claim 31, further comprising:
receiving, by the electronic device, a selection of an operational environment; and
controlling, by the electronic device, the laser based on the operational environment.

33. The method of claim 32, further comprising:
receiving, by the electronic device, a selection of a mode of use; and
controlling, by the electronic device, the laser based on the mode of use.
